(19) United States
(12) Patent Application Publication
(10) Pub. No.: US 2011/0030449 A1
(43) Pub. Date: Feb. 10, 2011

(54) SOIL CONTAMINATION DETECTOR AND DETECTION METHOD

(75) Inventors: Takehisa Hosono, Nagano, (JP); Naoki Urushihata, Tokyo (JP); Hideki Tanemura, Tokyo (JP); Yukinobu Tajima, Tokyo (JP); Shuuji Hirohama, Tokyo (JP)

Correspondence Address: CANTOR COLBURN LLP, 20 Church Street, 22nd Floor, Hartford, CT, 06103, US

(73) Assignee: LAND ECO CORPORATION, Nagano, (JP)

(21) Appl. No.: 12/092,517

(22) PCT Filed: Oct. 31, 2006
(86) PCT No.: PCT/JP2006/321691

§ 371 Date: Sep. 21, 2009

(30) Foreign Application Priority Data
Nov. 2, 2005 (JP) 2005-319900

(51) Int. Cl. G01N 33/24

(52) U.S. Cl. 73/23.34

(57) ABSTRACT

The soil contamination detector and detection method are provided, the detector and the detection method can significantly simplify investigation and analysis of a contaminant without use of any large analyzer like a gas chromatograph. The soil contamination detector comprises a sensor (10) and a control mechanism (12). The sensor (10) is disposed in a region (4) as a contamination investigation object, for detecting odor (S) of a substance (M) contaminating soil (G). The control mechanism (12) compares the concentration (D) of the contaminant (M) detected by the sensor (10) with the tolerance limit concentration (Pd) of the contaminant (M) to determine the contamination.

### TECHNICAL FIELD

The present invention relates to a soil contamination detector and a detection method, and in particular to a device and a method for detecting a contaminant based on an odor of a substance contaminating a soil.

### BACKGROUND ART

Soil that is contaminated by hazardous substances may adversely affect the health of residents, and is therefore unsuitable as a place of residence and also undesirable for raising animals or growing plants. For the above-described reasons, or due to various other factors, the fact that the soil is contaminated by hazardous substances constitutes a factor (a defect factor) that reduces the collateral value as real estate.

Heretofore, soil contamination investigation for investigating whether or not soil contains such hazardous substances is performed by boring the soil to be investigated, collecting sample lots at various depths, transporting the collected sample lots to an investigation institution (investigation facility) by automobile or the like, and quantitatively analyzing various types of hazardous substances using a gas chromatograph or other analyzer in the investigation institution (investigation facility).

However, such a conventional method requires very cumbersome work, such as boring the soil to be investigated, collecting sample lots at various depths, transporting the collected sample lots to an investigation institution, and quantitatively analyzing them using a gas chromatograph or the like. Therefore, there is a problem in that the efforts and costs will become enormous.

To address such a problem, although there is a demand for techniques capable of easily investigating soil contamination, currently, soil contamination investigation techniques which can satisfy this demand have not been achieved.

As another conventional technique, there is proposed a system for providing environmental data in order to precisely ascertain the environment of real estate (see Patent Publication 1).

However, although the proposed system lists odors and chemical substances as the environmental data, the publication does not specifically propose detection of soil contaminants. Therefore, it does not solve the above-described problem of conventional techniques.

Patent Publication 1: Japanese Laid-Open Patent Publication No. JP 2004-185275 A

### DISCLOSURE OF INVENTION

### Problems to be Solved by the Invention

The present invention was proposed to address the above-described problem of conventional techniques, and an object of the present invention is to provide a soil contamination detector and a detection method that can significantly simplify investigation and analysis of a contaminant without use of any large analyzer like a gas chromatograph.

### Means for Solving the Problems

As a result of various research, the inventors have found that when it is possible to analyze whether or not soil is contaminated by a hazardous substance based on various types of odors generated in the soil to be inspected, soil contamination can be determined in the field without making great efforts to collect samples, and without transporting the samples to an investigation institution. The present invention has been proposed based on the above findings.

According to one aspect of the present invention, there is provided a soil contamination detector comprising a sensor (10) disposed in a region (4) under contamination investigation for detecting an odor (S) of a substance (M) contaminating soil (G); and a control mechanism (control unit) (12) to compare a concentration (D) of a contaminant (M) detected by the sensor (10) with a tolerance limit concentration (Pd) of the contaminant (M) to determine contamination (claim 1).

Further, according to another aspect of the present invention, there is provided a soil contamination detection method comprising disposing a sensor (10) (on a surface of soil or on the ground level) in a region under contamination investigation to detect an odor (S) of a contaminant (M); and comparing a concentration (D) of a contaminant (M) detected by the sensor (10) with a tolerance limit concentration (Pd) of the contaminant (M) to determine contamination (claim 4).

In the present invention, a sensor which reacts to (can detect) each of a plurality of different types of contaminants on a one-to-one basis (for example, a thin film sensor) is prepared as the above-described sensor (thin film sensors or similar type sensors are prepared in a number corresponding to the number of types of contaminants).

Alternatively, a plurality of sensors may be combined to make determination based on their output pattern (radar chart).

Here, a thin film sensor as described above is produced by colliding a gas of a substance to be detected against a thin film having a molecular-level thickness.

By colliding a gas of a substance to be detected against a thin film as described above, molecular-level holes (or molecular-level lattice defects) are formed in this thin film. Such holes have a molecular-level shape identical to that of the substance to be detected, and therefore only the substance to be detected can pass through the holes, or, in other words, can pass through the thin film.

When a molecule of the substance to be detected passes through the thin film, the molecule collides against, for example, a power generation element disposed on the back side of the thin film, and generates an electrical signal.

In the present invention, the substance to be detected may include, for example, cadmium (Cd), lead (Pb), hexavalent chromium, cyanogen compounds, arsenic, selenium, mercury, alkyl mercury compounds, PCB, organophosphorus compounds, thiuram, simazine, thiobencarb, and other heavy metals, and dichloromethane, carbon tetrachloride, 1,2-dichloroethane, 1,1-dichloroethane, cis-1,2-dichloroethylene, 1,1,1-trichloroethane, 1,1,2-trichloroethane, trichloroethylene, tetrachloroethylene, 1,3-dichloropropene, benzene, and other volatile organic compounds.

According to still another aspect of the present invention, it is preferable that, in the soil contamination detector, the sensor (10) is configured to be capable of being inserted into a borehole (6) drilled in the region (4) under contamination investigation, and capable of moving in the borehole (6) and detecting an odor (S) of a contaminant (M) at a predetermined depth, and an odor (S) coming up from below the predetermined depth is blocked from reaching the sensor (10) (claim 2).

Further, according to still another aspect of the present invention, it is preferable that the soil contamination detection method further comprises drilling a borehole (6) in the region under contamination investigation; inserting the sensor (10) into the borehole (6); and stopping the sensor (10) at a predetermined depth and blocking an odor (S) coming up from below the predetermined depth to detect an odor (S) generated from a soil at the depth (claim 5).

Further, according to still another aspect of the present invention, there is provided a soil contamination detector comprising a sensor (10A) disposed in a hole (a borehole 6; including a groove or a relatively large region) drilled in a region (4) under contamination investigation, the hole being filled with water (W), wherein the sensor (10A) is configured to detect a contaminant (such as a heavy metal) dissolved in the water within the hole (6); and a control mechanism (control unit) (12) for comparing a concentration (D) of a contaminant detected by the sensor (10) with a tolerance limit concentration (Pd) of the contaminant to determine contamination (claim 3).

Further, according to still another aspect of the present invention, there is provided a soil contamination detection method comprising drilling a hole (a borehole 6) in a region under contamination investigation; immersing a sensor (10A) in water (W) which has been filled into the drilled hole (6); detecting a contaminant (such as a heavy metal) dissolved in the water using the sensor (10); and comparing a concentration (D) of the detected contaminant with a tolerance limit concentration (Pd) of the contaminant (M) to determine contamination (claim 6).

### Advantages of the Invention

According to the present invention, which comprises the above-described features, because it is configured such that contamination is determined by using a sensor disposed in a region under contamination investigation, and comparing a concentration of a contaminant detected by the sensor with a tolerance limit concentration of the contaminant (claims 1 and 3), soil contamination can be determined simply by having a structure for transmitting an output from the sensor to the control mechanism.

According to the present invention having such a structure, soil contamination can be determined far more easily than the case where samples are drilled, transported to an analysis facility, and subjected to gas chromatography analysis at the analysis facility. Further, cost reduction can be achieved by eliminating the necessity for collecting samples, transporting them, and processing them in a special-purpose facility.

Because studies of the inventors have indicated that it is possible to identify all odors of contaminants currently known as causes of soil contamination problems, detection of an odor makes it possible to very precisely detect a contaminant. In addition, because it is also possible to determine a concentration of a contaminant during detection of an odor, not only qualitative investigation regarding the presence or absence of a contaminant but also quantitative investigation regarding the concentration of the contaminant can be performed.

When the present invention further comprises drilling a borehole in the region under contamination investigation; inserting the sensor into the borehole; and stopping the sensor at a predetermined depth and blocking an odor coming up from below the predetermined depth to detect an odor generated from a soil at the depth (claims 2 and 4), because contamination investigation can be performed by detecting odors from the soil at each predetermined depth, vertical-direction contamination distributions or other contamination conditions can be ascertained.

When the present invention further comprises using a sensor (10A) configured to detect a contaminant (such as a heavy metal) dissolved in water, soil contamination can be determined by immersing the sensor (10A) in water in which a contaminant (such as a heavy metal) present in the region under contamination investigation is dissolved (for example, in water which has been filled into the borehole 6).

### BEST MODE FOR CARRYING OUT THE INVENTION

Embodiments of the present invention will be described below with reference to the accompanying drawings.

FIGS. 1 through 3 illustrate a first embodiment of the present invention.

Referring to FIG. 1, which illustrates an overall structure, a plurality of thin film sensors 10a, 10b . . . 10y (hereinafter, collectively referred to as 10) are provided, in a number corresponding to the number of types of contaminants to be detected, over a ground level GL of a ground G within a region 4 under contamination investigation in order to analyze odors S wafting up from the ground level GL, and the plurality of sensors 10 are connected through a signal line L10 to a control mechanism 12, which will be described in detail later. Here, the signal line L10 is formed by binding signal lines La, Lb . . . Ly for communicating detection data transmitted from each of the sensors 10.

The sensors 10 can detect the presence or absence of a contaminant, and can also detect its concentration D.

When the thin film sensors described above are used, if the concentration D of a contaminant to be detected is higher (thicker), the number of molecules passing through the thin film becomes larger, and the strength of a generated signal is increased. Therefore, by detecting the strength or amplitude of a detection signal, the concentration of contamination can also be measured, and it can be determined whether or not the concentration of the particular contaminant exceeds various types of criteria.

The control mechanism 12 is connected through a signal line L12 to a display 18 serving as display means.

FIG. 2 illustrates a block structure of the control mechanism 12.

The control mechanism 12 indicated by a dotted line in FIG. 2 includes concentration determination means (determiner) 14 for determining a concentration D of each contaminant M, comparison means (comparator) 16 for comparing the concentration D with a threshold value, and storage means (for example, a database) 20 for storing the determined concentration D of a contaminant, various types of threshold values, results of comparison, and the like.

The concentration determination means 14 is connected with the output signal line L10 of the sensors 10 disposed within the region 4 under contamination investigation, and receives input of data for each type of detection substance from each of the plurality of sensors 10. The concentration determination means 14 is connected to the storage means 20 through signal lines L14a and L20a.

Here, a detection signal from each sensor is transmitted through the signal line L14a from the concentration determination means 14 to the storage means 20. Characteristics of a sensor output signal and a contaminant concentration are transmitted through the signal line L20a from the storage means 20 to the concentration determination means 14.

Further, the concentration D determined by the concentration determination means 14 is transmitted to and stored in the storage means 20 through a signal line L14c and a signal line L14b branching off therefrom.

The comparison means 16 is connected with the concentration determination means 14 through the signal line L14c, and is connected to the storage means 20 through signal lines L20b and L16.

Here, threshold value data for contaminant concentrations is transmitted through the signal line L20b from the storage means 20 to the comparison means 16. Results of comparison determined by the comparison means 16 are transmitted to the storage means 20 through the signal line L16.

The storage means 20 is further connected to the display 18 serving as display means through signal lines L18 and L20c. Here, information processed by the control mechanism 12 is selected as desired using an external display terminal, and is displayed on the display 18.

The display 18 is connected through a signal line L36 to operating means 36 which can select, as desired, the information to be displayed on the display 18.

It should be noted that the display 18 in the illustrated example provides image display, but may be any other known means such as a handy printer or display on a mobile phone.

FIG. 3 is a flowchart illustrating control of a soil contamination detector having the above-described structure.

Actions taken according to the first embodiment will be described with reference to FIGS. 1 and 2 described above, and following the steps (flow) shown in FIG. 3.

As described above, the sensors 10 disposed over the ground level GL of the ground G within the region 4 under contamination investigation (the plurality of sensors 10 provided in a number corresponding to the number of types of contaminants to be detected) detect odors wafting up from the ground level GL, and produce output signals. At step in FIG. 3, it is determined whether or not an output signal from a sensor 10 is received by the concentration determination means 14 of the control device 12 (step S1).

If the concentration determination means 14 does not receive any output signal from the sensors 10 (“no” at step S1), the operation proceeds to a reception waiting state (a loop in which the determination at step S1 is “no”).

If an output signal from a sensor 10 is received by the concentration determination means 14 (“yes” at step S1), a concentration D of a detected contaminant is determined based on the characteristics of a sensor output signal and a contaminant concentration stored in the storage means 20 (step S2).

After the concentration D is determined (step S2 is completed), the concentration D is compared with a threshold value which is a tolerance value determined based on various types of criteria (step S3). Then, a result of the comparison is stored in the storage means 20 (step S4).

Next, it is determined whether or not steps S1 through S4 are performed for all contaminants to be detected (for all types of contaminants) (step S5).

If steps S1 through S4 are not performed for all types of contaminants (“no” at step S5), the operation returns to step S1. On the other hand, if steps S1 through S4 are performed for all types of contaminants (“yes” at step S5), the operation proceeds to step S6. In this step, results of steps S1 through S4 (such as a concentration D and a result of comparison with a threshold value) for all types of contaminants are stored in the storage means 20, and can be externally accessed (or referred to).

At step S6, it is determined whether or not data for a concentration D of a particular contaminant is to be displayed. If data for a concentration D is not to be displayed (“no” at step S6), it is not displayed on the display 18, and the operation is terminated bypassing step S7.

On the other hand, if data for a concentration D is to be displayed (“yes” at step S6), the data is transmitted to the display 18 (step S7), and the data for a concentration D is displayed.

The display 18 can provide data display for a selected particular contaminant. To switch from data for a contaminant being displayed to data display for another contaminant, an instruction may be provided through the operating means 36 (FIG. 2).

When display on the display 18 is completed for contaminants which need to be displayed, a series of operations described with reference to FIG. 3 is completed.

It should be noted that by successively detecting a concentration D of a contaminant over a certain period of time, development of soil contamination can be monitored as changes occurring over time. Therefore, for example, when a concentration has increased sharply, issuance of an alert or other necessary measures can be performed.

FIG. 4 illustrates a modification example of the above-described first embodiment.

A plurality of boreholes 6 are drilled in an investigation target region 4. Here, as in the first embodiment, a plurality of sensors are disposed over a ground level GL near the drilled boreholes 6 (the sensors are not shown in this figure).

With the structure as shown in FIG. 4, odors S of contaminants waft up via the boreholes 6 drilled through the contaminated region 4. The odors S wafting up via the boreholes 6 drilled through the contaminated region 4 are thicker than the odors S wafting up from the ground level GL as shown in FIG. 1, and a concentration of a contaminant contained in the odors S wafting up via the boreholes 6 is high. Therefore, compared with the method as shown in FIG. 1 (an investigation method based on the odors S wafting up from the ground level), the method as shown in FIG. 4 (an investigation method based on the odors S wafting up via the boreholes 6) provides higher accuracy for determining the presence or absence of a contaminant and for detecting a concentration D.

Also in the modification example illustrated in FIG. 4, as in the first embodiment in FIGS. 1 through 3, because there is no necessity for collecting samples, transporting them, and processing them in a special-purpose facility, costs can be correspondingly reduced.

Further, also in the modification example in FIG. 4, successive changes of soil contamination can be observed over time.

In the modification example in FIG. 4, when the boreholes 6 are left standing for a long period of time after they are drilled, the edge of a borehole 6 may collapse and bury the borehole 6.

To avoid this, by filling the boreholes 6 with a nonwoven fabric or a porous material, or by reinforcing the inner walls of the boreholes 6 with perforated metal in which perforations are formed by pierce punching, the filler or the perforated metal will prevent the boreholes 6 from being buried due to the collapse.

Simultaneously, through a plurality of through holes of the perforated metal, or through continuous clearance (space) in the nonwoven fabric or the porous material, it can be ensured that odors S of a contaminant M contained in the soil are allowed to waft up in the boreholes 6.

Also in this modification example, the structure can be considered as a soil contamination alert device.

FIGS. 5 through 11 illustrate a second embodiment.

The ground in Japan is mostly composed by layering a plurality of different types of layers, and also in connection with soil contamination it can be expected that the conditions of contamination may differ at different depths.

In such situations, in the first embodiment shown in FIGS. 1 through 4, the conditions of contamination varying at different depths of the ground G cannot be ascertained.

According to the second embodiment illustrated in FIGS. 5 through 11, the state of contamination is investigated by detecting odors S from the ground G for each predetermined depth, and therefore this embodiment has an advantage in that the conditions of contamination varying at different depths can be ascertained.

FIG. 5 illustrates a step of drilling a borehole 6 into the ground G which is to be under contamination investigation. There is shown a state in which the borehole 6 is drilled to a predetermined depth using a boring rod 30 to the front end of which a boring bit 32 is attached.

FIG. 6 illustrates a step of inserting a sensor 10 to a predetermined depth (“desired measurement depth”) in the borehole 6 drilled as shown in FIG. 5.

A signal line L10 communicating with a control mechanism 12 provided over the ground is connected to the sensor 10, and the sensor 10 can move freely up and down within the borehole 6 by means of, for example, a cable-like component (not shown; which is preferably a separate component different from a cable for the signal line). Thus, the contaminants M can be detected at all depths.

Here, in regards to the contaminants M present in depth ranges other than the depths of detection, especially when an odor S comes up from below the depths of detection, it is necessary to take measures to prevent detection of the odor S coming up from below. This is because it will be impossible to precisely detect how the contaminants are buried in the vertical direction if an odor coming up from below mixes with an odor present at a location where the sensor 10 is located.

FIG. 7 illustrates an example of measures which can be taken against such a situation.

In FIG. 7, a signal line L10 communicating with a control mechanism 12 provided over the ground is connected to the sensor 10 within the borehole 6, and an expandable and contractible rubber balloon-like packer 11 is attached below the sensor 10. An air supply line (not shown) for expanding the packer 11 is connected to the packer 11, and the air supply line is bound together with the signal line L10.

FIG. 7 illustrates a state in which the packer 11 is expanded, and the expanded packer 11 comes into contact with the inner wall of the borehole 6 to separate and seal between an upper area and a lower area relative to the packer 11 in the vertical direction. Because the packer 11 seals, odors Su of the contaminants M wafting up from below the sensor 10 do not reach the sensor 10.

As a result, the sensor 10 can measure only odors So generated from the contaminants M present in the ground G at a depth where the sensor 10 is located, and flowing toward over the ground through the borehole 6.

FIG. 8 illustrates a state in which the sensor 10 and the packer 11 are being moved in order to collect odors S at, for example, a location lower than the depth illustrated in FIG. 7.

The packer 11 in the expanded state (FIG. 7) contacts the inner wall of the borehole 6 and seals odors wafting up from the lower area, and in this state, the packer 11 and the sensor 10 cannot be moved up or down. For this reason, in FIG. 8, air contained in the packer 11 is released over the ground through the air supply line, which is not shown, to cause the packer 11 to contract, thereby allowing the sensor 10 and the packer 11 to move up or down.

In the state illustrated in FIG. 8 (in which the packer 11 is contracted), the sensor 10 is moved, for example, downward in the vertical direction (in the direction of the arrow Z) to a predetermined depth. Then, the packer 11 is expanded again (the state in FIG. 7), and odors of contaminants are measured and detected.

FIG. 9 is a flowchart illustrating a flow of actions of a soil contamination detector having the above-described structure.

With reference to FIGS. 5 through 8 described above, the respective steps in FIG. 9 will be described below. It should be noted that the flowchart in FIG. 9 describes the sensor 10 (FIGS. 5 through 8) as “sensor head”.

First, a borehole 6 is drilled into the ground G within a region under contamination investigation (see FIG. 5; step S11 in FIG. 9).

Subsequently, a sensor 10 is inserted into the borehole 6 to a desired depth (see FIG. 6; step S12 in FIG. 9).

Here, the term “desired depth” refers to a depth at which it is necessary to detect odors S generated from the contaminated ground G.

When the sensor 10 has reached a desired depth, it stops at that depth, and the packer 11 is expanded (see FIG. 7; step S13 in FIG. 9).

The packer 11 is expanded in order to block odors Su coming up from the lower area.

In the state illustrated in FIG. 7 (at the desired depth), odors So of various types of contaminants coming from the wall of the borehole 6 are detected by the sensor 10 (a loop including steps S14 and S15 in FIG. 9, in which the determination at step S15 is “no”).

The sensor 10 performs the above-described measurement and detection throughout all depths of the borehole 6 (a loop in which the determination at step S16 is “no”), and when the detection is completed throughout all depths (“yes” at step S16), the operation is completed.

FIG. 10 illustrates a modification example of the second embodiment.

In the manner of detection illustrated in FIGS. 7 and 8, the packer 11 is expanded to thereby block odors wafting up from below the position of detection. In contrast, according to the modification example in FIG. 10, the sensor 10 is disposed within a hollow casing 24.

Here, the casing 24 has a plurality of holes 26 that are formed in a circumferential wall 25 facing the inner wall of the borehole 6, but does not have any through holes formed in upper and lower walls or at least in the bottom wall, and provides blockage.

When odors are measured using the casing 24, odors generated from the soil at a desired measurement depth enter the casing 24 through the holes 26 formed in the circumferential wall 25. Thus, the odors are detected by the sensor 10.

On the other hand, odors Su wafting up from the soil located below the desired depth are blocked by the bottom of the casing 24, and are therefore prevented from being detected by the sensor 10 disposed within the casing 24.

In other words, because the odors Su wafting up from the soil in the lower area are blocked by the bottom of the casing 24, mixing with odors So generated from the soil at the desired depth within the casing 24 is prevented, and a decrease in accuracy of detecting the odors So generated from the soil at the desired depth is prevented.

Also in the modification example in FIG. 10, as in FIGS. 7 and 8, a cable-like component (not shown) for causing the sensor 10 and the casing 24 to move up and down is provided separately from the signal cable L10.

In FIGS. 5 through 9, the sensor 10 is moved up and down using a cable-like component, which is not shown, but the sensor may be moved up and down in a manner as illustrated in FIG. 11.

In FIG. 11, a rod 38 is inserted into the borehole 6, a guide rail 36 is provided in the rod 38, and a self-moving mechanism, which is not shown, is provided (known mechanisms can be used without modification).

The sensor 10 moves up or down along the guide rail 36 by means of the self-moving mechanism.

FIGS. 12 through 17 illustrate a third embodiment of the present invention.

The third embodiment is an embodiment which combines the first embodiment and the second embodiment. The third embodiment will also be described with reference to FIGS. 1 and 2 of the first embodiment as the overall structure and the control mechanism are generally similar to those described in the first embodiment.

In the third embodiment, first, as shown in FIG. 12, a plurality of boreholes are drilled as evenly as possible throughout the region 4 to be subjected to a contamination investigation.

Next, as shown in FIG. 13, odors of contaminants present in the ground G are measured at predetermined depths for each borehole 6 in a manner similar to those described with reference to FIGS. 5 through 11.

Then, the results obtained by measuring odors of contaminants present in the ground G at predetermined depths for each borehole 6 are input to the control mechanism 12 through the signal line 10 (FIG. 14).

Although it is not clearly shown in FIG. 14, the control mechanism 12 has therein a structure as shown in FIG. 2 of the first embodiment, in which a concentration D of an odor S is determined, it is compared with a threshold value, and the concentration D, results of comparison, and other data are stored in the storage device 20.

Here, although it is not illustrated in FIG. 14, the control mechanism 12 is connected to the display 18, as shown in FIGS. 1 and 2. The display 18 is configured to be able to display all of a plurality of different types of contaminants M to be investigated.

As the information to be displayed, the display 18 can collectively display all of the plurality of contaminants as a “contamination”, and can also display a distribution, a concentration D, and the like for each individual contaminant.

Using gradation on the display screen, the display can indicate the concentration of a contaminant to be displayed.

Further, for each individual contaminant, the display can display its distribution, concentration, and the like.

Alternatively, it is possible to display, for example, depth-direction distributions as shown in FIG. 15, displaying a distribution of a contaminant in a particular cross section. For example, FIG. 15 illustrates a state in which a layer of Pb (lead) Gp is present at a depth Xo, and a layer of Cd (cadmium) Gc is present at a depth X1, as is displayed on the display.

To two-dimensionally display a state of distribution for each contaminant, it is possible to employ a manner as shown in FIGS. 16 and 17, for example.

Here, FIG. 16 shows a lead distribution region Gp within the region 4 which has been under soil contamination investigation. Further, FIG. 17 shows a cadmium distribution region Gc within the region 4 which has been under soil contamination investigation.

The screen as shown in FIG. 15 and the screens as shown in FIGS. 16 and 17 can be switched by switching operation of the operating means 36.

It should be noted that also in FIGS. 16 and 17, the concentration of a contaminant can be indicated by gradation on the screen.

Various representations as shown in FIGS. 15 through 17 can be achieved by storing a concentration D, a result of comparison, and other data in the storage device 20 (see FIG. 14), and processing the data stored in the storage device 20 through a known information processing technique. However, in order to avoid complexity of the description, explanation of such known information processing techniques is omitted here.

FIG. 18 illustrates a fourth embodiment of the present invention.

According to the embodiments illustrated in FIGS. 1 through 17, a contaminant vaporized or diffused from the soil into the air is detected as an odor to determine the presence or absence of contamination or a degree of contamination. On the other hand, according to the fourth embodiment in FIG. 18, a contaminant (such as, a heavy metal) dissolved in water is detected by a sensor 10A to determine contamination.

The fourth embodiment will be described below with reference to FIG. 18, the description focusing on the difference from the first through third embodiments.

As shown in FIG. 18, a borehole 6 is filled with water W. The sensor 10A is immersed (in other words, “completely dipped”) in the water W.

When the ground G in which the borehole 6 is drilled is contaminated by, for example, a heavy metal, the heavy metal will be dissolved into the water W which fills the borehole 6. The sensor 10A detects the contaminant dissolved in the water W (in this case, the heavy metal), and outputs a detection signal to over-the-ground equipment, which is not shown in this figure, (for example, to the control device 12 and the display 18 shown in FIG. 1).

Here, in order to implement the fourth embodiment, after the borehole 6 is drilled into the ground G, water (for example, clean water) is pumped into the borehole 6 by means of equipment not shown, and the sensor 10A is immersed therein.

An alternative is, after the borehole 6 has been drilled into the ground G, to wait until discharged groundwater fills the borehole 6, and the sensor 10A may be immersed after the groundwater fills the borehole 6.

When the ground G is contaminated by, for example, a heavy metal, the heavy metal is dissolved into the clean water or groundwater which has been filled into the borehole 6, the dissolved heavy metal is detected by the sensor 10A, and contamination is determined. Further, when groundwater is used, it is possible to determine contamination of the groundwater in itself.

Other structure, operation, and advantages of the fourth embodiment in FIG. 18 are similar to those of the embodiments illustrated in FIGS. 1 through 17.

The illustrated embodiments are provided by way of example only, and the description is not intended to limit the technical scope of the present invention.

For example, the present invention can be applied as a technique for detecting odors S of sulfur (Sul) from the ground G, thereby locating a hot spring (a hot spring survey technique).

Further, it is also possible to provide a structure in which an alert is issued by monitoring changes of a contaminant M over time.

Although the illustrated embodiments are configured to be able to detect all substances under contamination investigation, it is also possible to provide a structure in which only a representative contaminant can be detected.

Further, although in the illustrated embodiments, a plurality of sensors each reacting to only one type of contaminant, such as thin film sensors, are provided (in a number corresponding to the number of types of substances to be detected), it is also possible to adopt a sensor system of a type in which a plurality of sensors 10 each reacting to a plurality of contaminants M are combined to create a radar chart-like pattern to identify a particular contaminant M based on this pattern.

### BRIEF DESCRIPTION OF DRAWINGS

FIG. 1 is a block diagram illustrating an overall structure of a first embodiment of the present invention.

FIG. 2 is a block diagram of a control device shown in FIG. 1.

FIG. 3 is a flowchart illustrating control according to the first embodiment.

FIG. 4 illustrates a modification example of the first embodiment.

FIG. 5 illustrates a step of drilling a borehole according to a second embodiment.

FIG. 6 illustrates a step of inserting a sensor into the borehole according to the second embodiment.

FIG. 7 illustrates a step of expanding a packer according to the second embodiment.

FIG. 8 illustrates a step of contracting a packer according to the second embodiment.

FIG. 9 is a flowchart illustrating a flow of actions according to the second embodiment.

FIG. 10 illustrates a state in which a casing is used instead of the packer.

FIG. 11 illustrates an embodiment in which a rod with a sensor moving along a guide rail is inserted into the borehole.

FIG. 12 illustrates a state in which a plurality of boreholes are drilled according to a third embodiment.

FIG. 13 illustrates a state in which a borehole is drilled and a sensor is inserted according to the third embodiment.

FIG. 14 illustrates a state in which data obtained at each depth of each borehole is transmitted to a control device according to the third embodiment.

FIG. 15 illustrates a depth-direction distribution of each contaminant according to the third embodiment.

FIG. 16 two-dimensionally illustrates a distribution state of lead.

FIG. 17 two-dimensionally illustrates a distribution state of cadmium.

FIG. 18 illustrates a fourth embodiment.

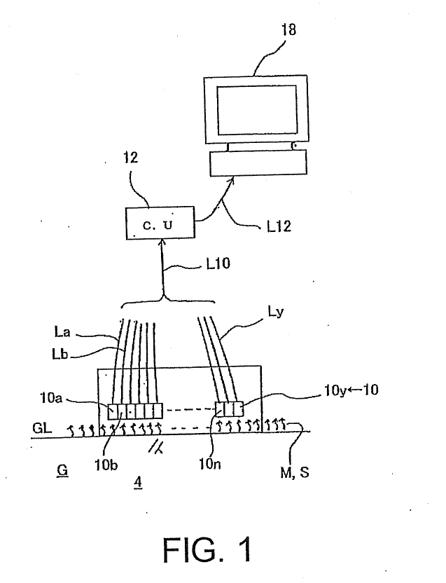

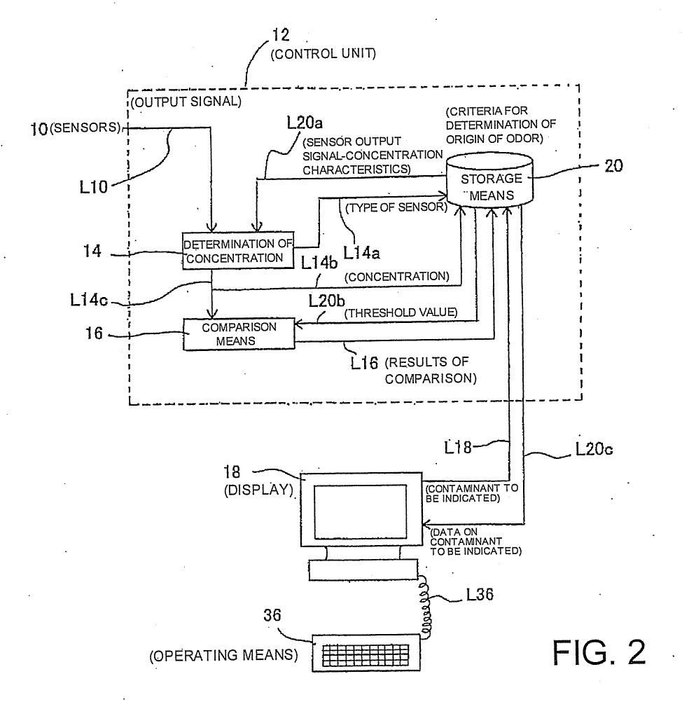

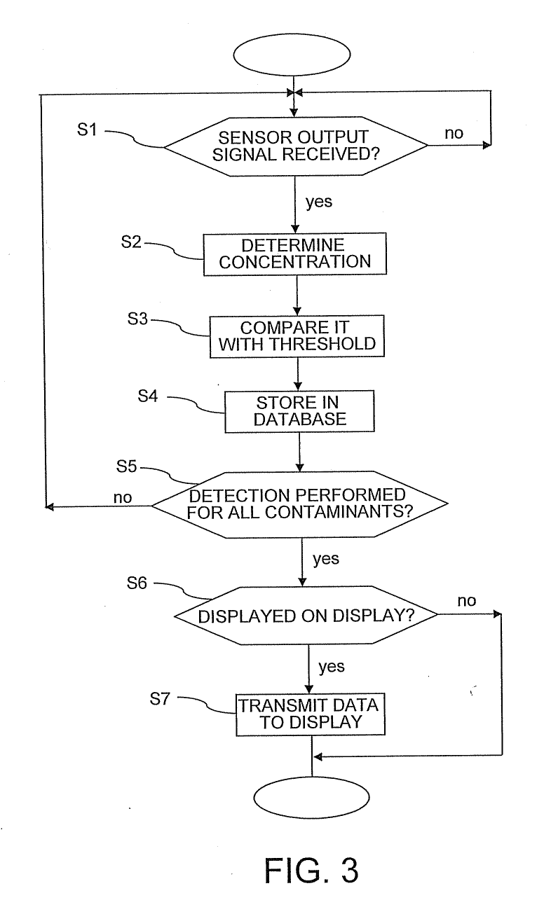

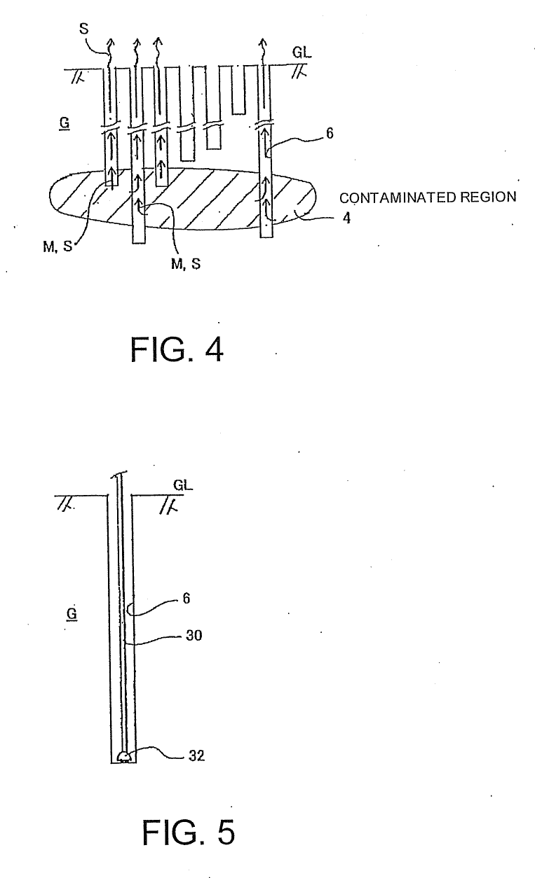

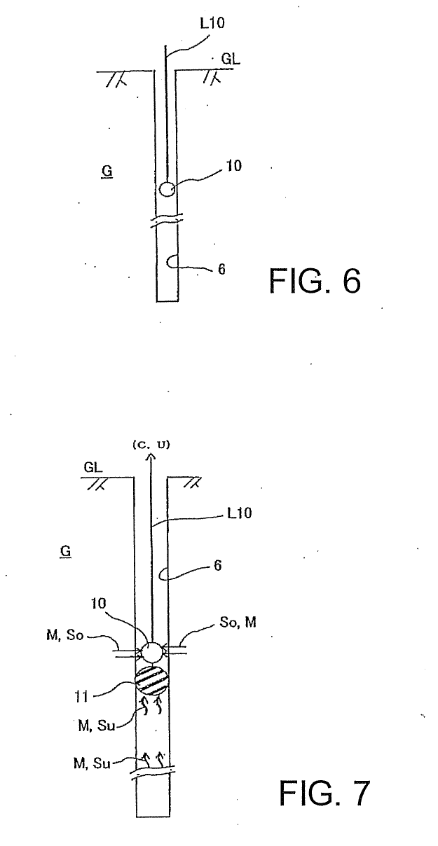

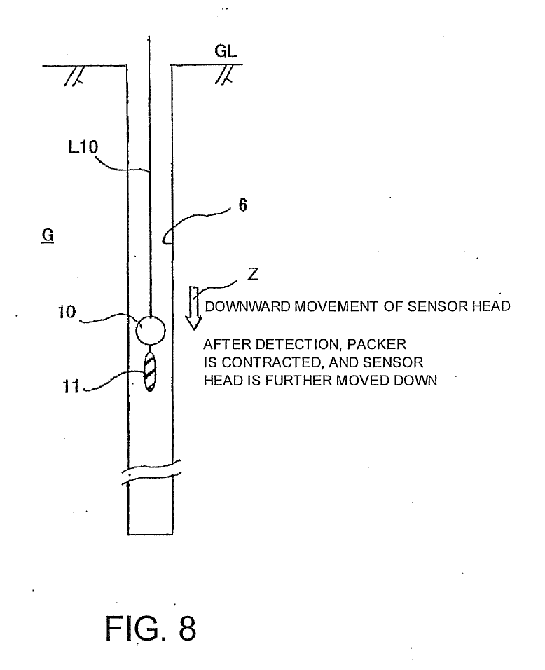

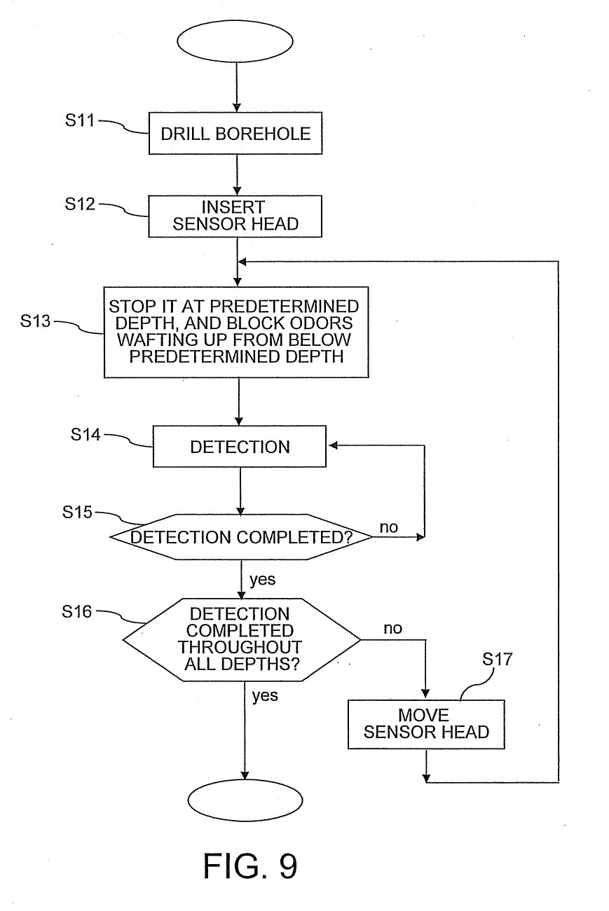

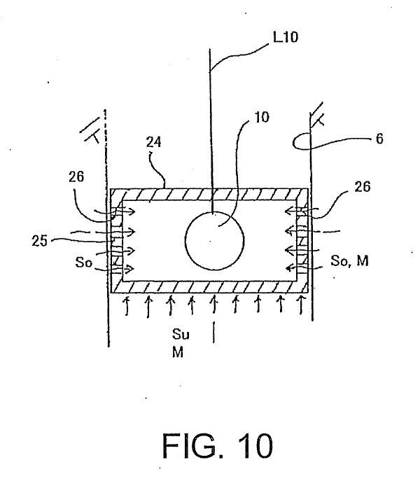

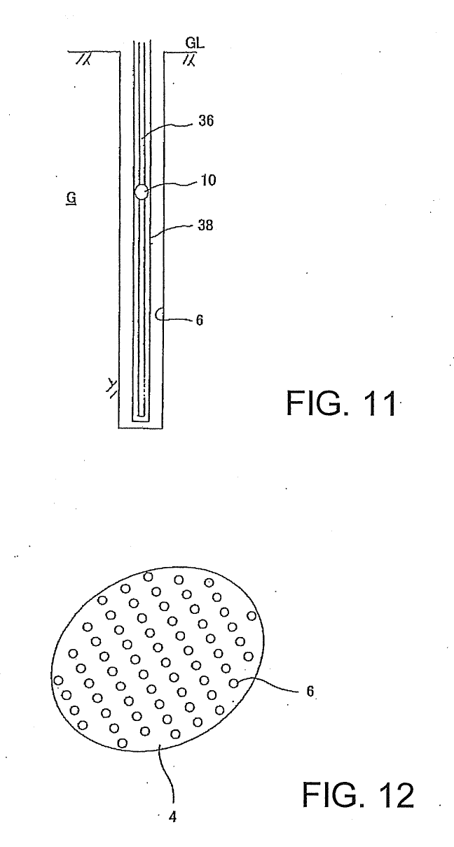

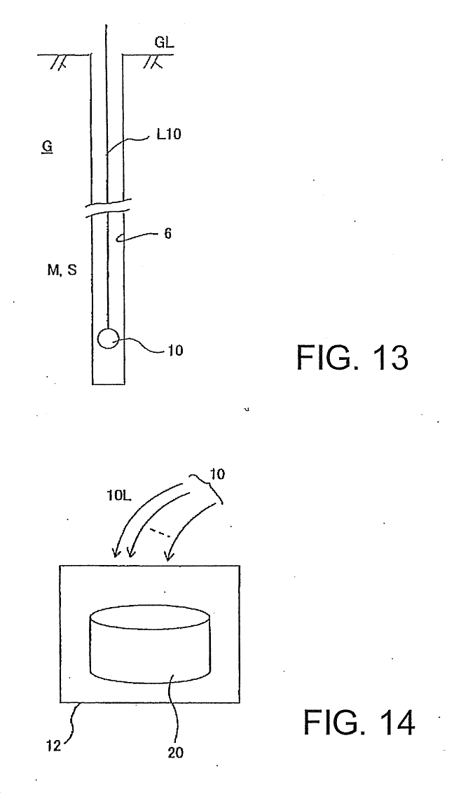

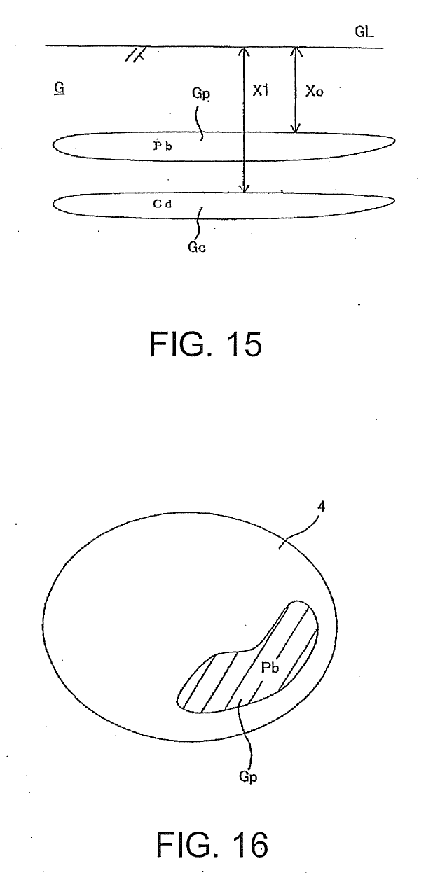

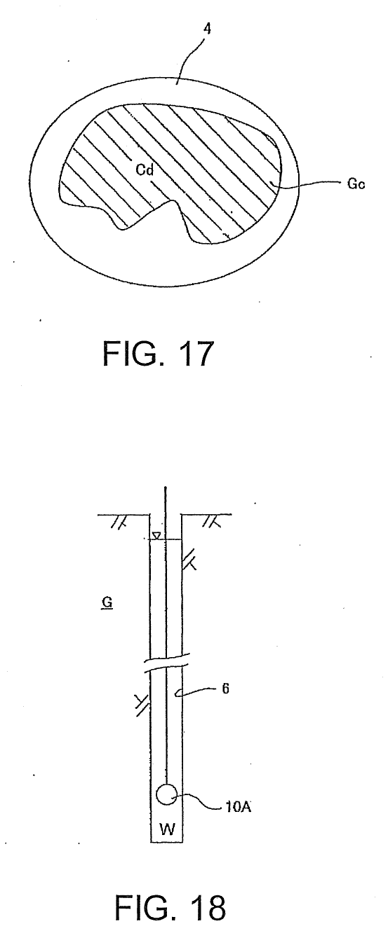

### EXPLANATION OF REFERENCE NUMERALS

D CONCENTRATION OF ODORS
G GROUND
M CONTAMINANT
S ODOR
4 REGION UNDER CONTAMINATION INVESTIGATION
6 BOREHOLE
10, 10A SENSOR
11 PACKER
12 CONTROL DEVICE
14 CONCENTRATION DETERMINATION MEANS
16 COMPARISON MEANS
18 DISPLAY MEANS, DISPLAY
20 STORAGE MEANS
36 OPERATING MEANS

1. A soil contamination detector apparatus, comprising:
a sensor disposed in a region under contamination investigation for detecting an odor of a substance contaminating a soil; and
a control mechanism for comparing a concentration of a contaminant detected by the sensor with a tolerance limit concentration of the contaminant to determine contamination.

2. The soil contamination detector apparatus according to claim 1, wherein
the sensor is configured to be capable of being inserted into a borehole drilled in the region under contamination investigation, and capable of moving in the borehole and detecting an odor of a contaminant at a predetermined depth, and
an odor coming up from below the predetermined depth is blocked from reaching the sensor.

3. A soil contamination detector apparatus, comprising:
a sensor disposed in a hole drilled in a region under contamination investigation, the hole being filled with water, wherein the sensor is configured to detect a contaminant dissolved into the water within the hole; and
a control mechanism for comparing a concentration of a contaminant detected by the sensor with a tolerance limit concentration of the contaminant to determine contamination.

4. A soil contamination detection method, comprising:
disposing a sensor in a region under contamination investigation to detect an odor of a contaminant; and
comparing a concentration of a contaminant detected by the sensor with a tolerance limit concentration of the contaminant to determine contamination.

5. The soil contamination detection method according to claim 4, the method further comprising:
drilling a borehole in the region under contamination investigation;
inserting the sensor into the borehole; and
stopping the sensor at a predetermined depth and blocking an odor coming up from below the predetermined depth to detect an odor generated from soil at the depth.

6. A soil contamination detection method, comprising:
drilling a hole in a region under contamination investigation;
immersing a sensor in water which has been filled into the drilled hole;
detecting a contaminant dissolved in the water using the sensor; and
comparing a concentration of the detected contaminant with a tolerance limit concentration of the contaminant to determine contamination.
